(19) United States
(12) Patent Application Publication
(10) Pub. No.: US 2015/0335365 A1
(43) Pub. Date: Nov. 26, 2015

(54) FIXATION DEVICE FOR A MAU-TYPE OSTEOTOMY PROCEDURE

(71) Applicant: NEUTIN ORTHOPEDICS, LLC, MILLERSVILLE, MD (US)

(72) Inventors: Steven Keith Neufeld, Washington, DC (US); Albert Eugene Austin, MILLERSVILLE, MD (US)

(21) Appl. No.: 14/722,081

(22) Filed: May 26, 2015

Related U.S. Application Data
(60) Provisional application No. 61/997,182, filed on May 24, 2014.

(51) Int. Cl. A61B 17/80

(52) U.S. Cl. CPC A61B 17/8061

(57) ABSTRACT

An internal plate fixation device is provided for load bearing and non-load bearing fixation for the Mau-type osteotomy procedure for hallux valgus correction in the first metatarsal bone of the foot. The preferred example includes a set design plate having screw holes for attachment of the plate to the first metatarsal bone of the foot. Screw holes are located on the plate, including screw holes on top of the plate and screw holes at the ends of the plate. A temporary small holding wire hole is located in the plate to hold the plate to the bone temporarily. This plate allows both angled screw fixation of the Mau osteotomy and locking screw hole capability to ensure stability when fixating the Mau osteotomy.

### CROSS-REFERENCE TO RELATED APPLICATIONS

The present application claims the benefit of U.S. Provisional Application No. 61/997,182, filed May 24, 2014, which is hereby incorporated by reference herein in its entirety.

### FIELD

This present disclosure relates generally to medical devices, and, more particularly, to fixation devices for a Mau-type osteotomy procedure.

### BACKGROUND

The Mau osteotomy was first used in 1915 and presented by Mau in 1926. Mau modified the Ludloff osteotomy cut by changing the direction of the osteotomy cut. The Ludloff osteotomy is a through-in-through, oblique diaphyseal osteotomy cut extending from dorsal proximal to plantar distal on the first metatarsal bone. Mau reversed the Ludloff cut and challenged the stability of the Ludloff osteotomy by creating a dorsal shelf to help resist weight-bearing forces. The Mau osteotomy is a through-in-through oblique diaphyseal osteotomy cut from plantar proximal to dorsal distal through the shaft of the first metatarsal.

The Mau osteotomy is made parallel to the weight bearing plane and its long dorsal shelf of bone supports the distal fragment, making dorsal displacement of the first metatarsal osteotomy cut unlikely. The Mau type osteotomy is an intrinsically stable metatarsal osteotomy cut because of the dorsal shelf that resists dorsal displacement forces. Weight bearing on the foot compresses the osteotomy.

The Mau osteotomy surgical technique is achieved with a standard medial approach incision to obtain exposure of the first metatarsal bone. After identifying the tarsometatarsal joint (TMTJ) and using a medical power saw, the oblique Mau osteotomy cut is begun about 1-2 centimeters from the first metatarsal head and made from dorsal-distal to proximal-plantar, parallel to the weight bearing surface of the first metatarsal bone. The plantar metatarsal bone fragment is then laterally rotated transversely and realigned to correct the desired anatomical alignment. Once appropriate realignment of the first metatarsal bone is completed to correct the hallux valgus deformity, various methods of fixation are inserted into the first metatarsal to hold the metatarsal in its realigned position.

Mau osteotomies have been previously fixated with K-wires and screws, but most notably always 2 or 3 screws. The fixation techniques reported vary in steps, screw diameter sizes used, k-wire sizes used, placement, etc.

### SUMMARY

In one or more embodiments, a medical fixation device comprises a straight section and first and second anatomically bended legs at opposite ends of the straight section. The straight section can comprise two or more screw holes. The anatomically bended legs can be thinner than the straight section, the thickness of the straight section decreasing at first and second transfer sections adjacent to the anatomically bended legs. The anatomically bended legs can be sized and shaped to encompass a tubular bone for fixation of a hallux valgus correction of a first metatarsal bone of a foot achieved by a Mau-type osteotomy procedure. The anatomically bended legs can each comprise a threaded screw hole.

In one or more embodiments, an internal plate fixation device for a Mau-type osteotomy procedure for hallux valgus correction in a first metatarsal bone of a foot comprises a middle section and anatomically bended legs at opposite ends of the middle section. The anatomically bended legs can be sized and shaped to encompass a tubular bone. The middle section can comprise two non-locking screw holes for fixation of the correction, the non-locking screw holes being configured to provide an angulation of screws to ensure screw engagement and fixation of the correction across an osteotomy site. The anatomically bended legs can each comprise one threaded screw hole for final fixation. The middle section can be thicker than the anatomically bended legs.

In one or more embodiments, a method comprises performing a Mau-type osteotomy cut of a bone to achieve a correction. The method can include implanting a fixation device to maintain the correction. The fixation device can comprise a middle section and anatomically bended legs at opposite ends of the middle section. The anatomically bended legs can be sized and shaped to encompass a tubular bone. The middle section can comprise two non-locking screw holes for fixation of the correction, the non-locking screw holes being configured to provide an angulation of screws to ensure screw engagement and fixation of the correction across an osteotomy site. The anatomically bended legs can each comprise one threaded screw hole for final fixation. The middle section can be thicker than the anatomically bended legs. The method can include inserting screws through the non-locking screw holes into the bone for initial fixation of the correction. The method can include, after initial fixation of the correction, adjusting the fixation device to the anatomy of the bone by bending down one or both of the anatomically bended legs. The method can include inserting screws through the threaded screw holes into the bone for final fixation of the correction.

In one or more embodiments, a method for fixation of a hallux valgus correction of a first metatarsal bone of a foot of a patient achieved by a Mau-type osteotomy procedure comprises performing the Mau-type osteotomy procedure to achieve a desired hallux valgus correction of the first metatarsal bone of the foot of the patient. A fixation device can be selected from a kit of fixation devices based on a characteristic of the patient and/or the desired correction. The kit can comprise two or more different sizes of fixation devices, and each size can be compatible with a respective predefined class of patient. Each fixation device can comprise a middle section and first and second legs at opposite ends of the middle section. The middle section can comprise two initial fixation screw holes. The middle section can comprise first and second transfer sections adjacent the first and second legs, respectively. The thickness of the device can be decreasing at each transfer section from a first thickness to a second thickness of the respective leg. The device can have an initial curvature compatible with its respective predefined class of patient. The legs can each comprise a threaded screw hole. The initial fixation screw holes can be non-locking screw holes adapted for angulation of non-locking screws to fixate the correction achieved by the Mau-type osteotomy procedure. The method can include applying the selected fixation device for initial fixation of the correction. After initial fixation of the correction, selected fixation device can be adjusting to the anatomy of the patient's bone by bending down one or both of the legs. The fixation device can have a variable thickness such that the fixation device is adapted to bend at portions oblique to a longitudinal axis of the fixation device to adjust to the anatomy of the patient's bone. Final fixation of the correction can be performed by applying screws through the threaded screw holes of the legs of the selected fixation device.

In one or more embodiments, a medical fixation device comprises a middle section and first and second legs at opposite ends of the middle section. The middle section can comprise two initial fixation screw holes. The middle section can comprise first and second transfer sections adjacent the first and second legs, respectively. The thickness of the device can decrease at each transfer section from a first thickness to a second thickness of the respective leg. The device can have an initial curvature compatible with a predefined class of patient for fixation of a hallux valgus correction of a first metatarsal bone of a foot achieved by a Mau-type osteotomy procedure. The legs can each comprise a threaded screw hole. The initial fixation screw holes can be non-locking screw holes adapted for angulation of non-locking screws to fixate the correction achieved by the Mau-type osteotomy procedure.

In one or more embodiments, a method, comprises performing an osteotomy cut of a bone of a patient to achieve a desired correction. A fixation device can be selected to maintain the correction based on a characteristic of the patient and/or the desired correction. The fixation device can comprise a middle section and legs at opposite ends of the middle section. The middle section can comprise two initial fixation screw holes. The legs can be sized and shaped to at least partially encompass a tubular bone. The middle section can comprise two non-locking screw holes for fixation of the correction. The non-locking screw holes can be configured to provide an angulation of screws to ensure screw engagement and fixation of the correction across an osteotomy site. The legs can each comprise one threaded screw hole for final fixation. The middle section can be thicker than the legs. The thickness of the device can be configured such that the device is adapted to bend at portions oblique to a longitudinal axis of the device. The method can include performing initial fixation of the correction with the selected fixation device. After initial fixation of the correction, selected fixation device can be adjusted to the anatomy of the bone by bending down one or both of the legs. Final fixation of the correction can be performed with the selected fixation device.

Objects and advantages of embodiments of the disclosed subject matter will become apparent from the following description when considered in conjunction with the accompanying drawings.

### BRIEF DESCRIPTION OF THE DRAWINGS

Embodiments will hereinafter be described with reference to the accompanying drawings, which have not necessarily been drawn to scale. Where applicable, some features may not be illustrated to assist in the illustration and description of underlying features. Throughout the figures, like reference numerals denote like elements. As used herein, various embodiments can mean one, some, or all embodiments.

FIG. 1 is a top plan view of an internal plate fixation device, according to one or more embodiments of the disclosed subject matter.

FIG. 2 is a top/oblique view of the plate fixation device shown in FIG. 1, according to one or more embodiments of the disclosed subject matter.

FIG. 3 is a back view of the plate fixation device shown in FIG. 1, according to one or more embodiments of the disclosed subject matter.

FIG. 4 is a side view of the plate fixation device shown in FIG. 1, according to one or more embodiments of the disclosed subject matter.

### DETAILED DESCRIPTION

The present disclosure provides an internal plate fixation device or technique. In one or more embodiments of the disclosed subject matter, the plate is adapted for the Mau osteotomy. In one or more embodiments, the internal plate fixation device or technique is adapted for non-load-bearing or load-bearing.

In some embodiments, the plate is adapted for osteotomy surgical correction, placement of the internal fixation plate, completion and fixation of the osteotomy correction, and final internal fixation of the osteotomy.

In one or more embodiments, the plate is specifically configured for implantation onto the first metatarsal bone during and after a Mau osteotomy hallux valgus correction to be completed. Fixation of the Mau osteotomy is performed with an anatomically configured and designed internal fixation plate designed for non-load-bearing or load bearing.

In some embodiments, the plate has non-locking screw fixation holes adapted for concentric angulation of non-locking screws to fixate the Mau osteotomy, both temporarily and for final fixation through the plate to both fixate the osteotomy and firmly hold the plate to the metatarsal bone.

In one or more embodiments, the plate has temporary fixation holes adapted for temporary fixation of the plate to the metatarsal bone before screw fixation.

In some embodiments, the plate has locking screw holes both distally and proximally, for different diameter locking screws, to allow for final fixation and to provide strength/stability of the osteotomy correction.

One advantage of embodiments shown and described herein is that precise fixation of the Mau osteotomy and both non-load-bearing and load-bearing are allowed. Another advantage of embodiments shown and described herein is that the plate will be able to accommodate a multitude of varied metatarsal anatomy sizes and metatarsal anatomy correction angles.

For purposes of promoting an understanding of the principles of the disclosed subject matter, reference will now be made to one or more examples illustrated in the drawings and described below. It is understood that no limitation to the scope of the disclosed subject matter is thereby intended. It is further understood that the present disclosure includes any alterations and modifications to the illustrated examples and includes further applications of the principles of the disclosed subject matter as would normally occur to one skilled in the art to which the disclosed subject matter pertains.

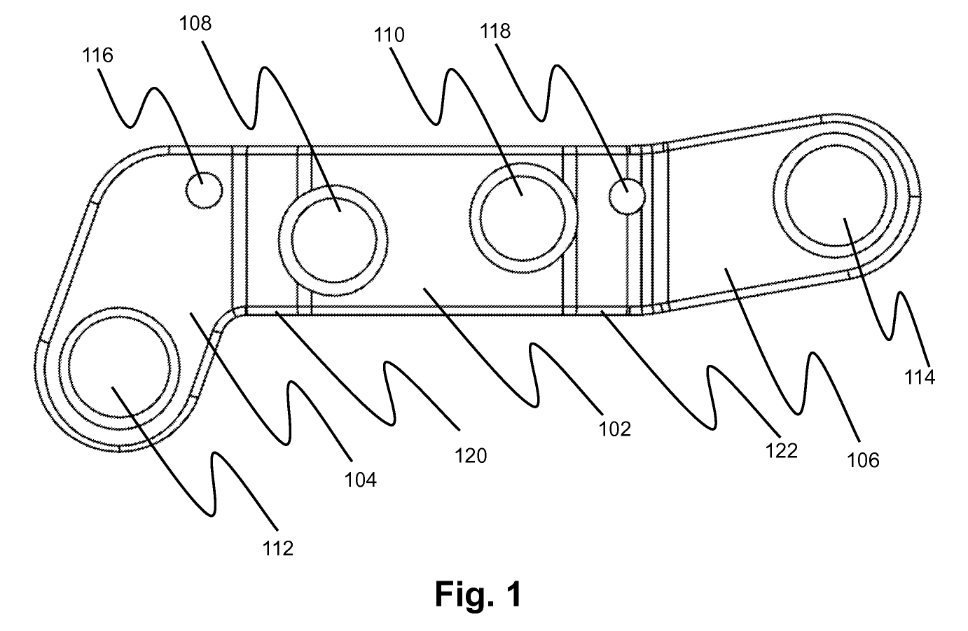

FIGS. 1-4 depict one or more embodiments of an internal plate fixation device for load bearing and non-load bearing fixation for the Mau-type osteotomy procedure for hallux valgus correction in the first metatarsal bone of the foot.

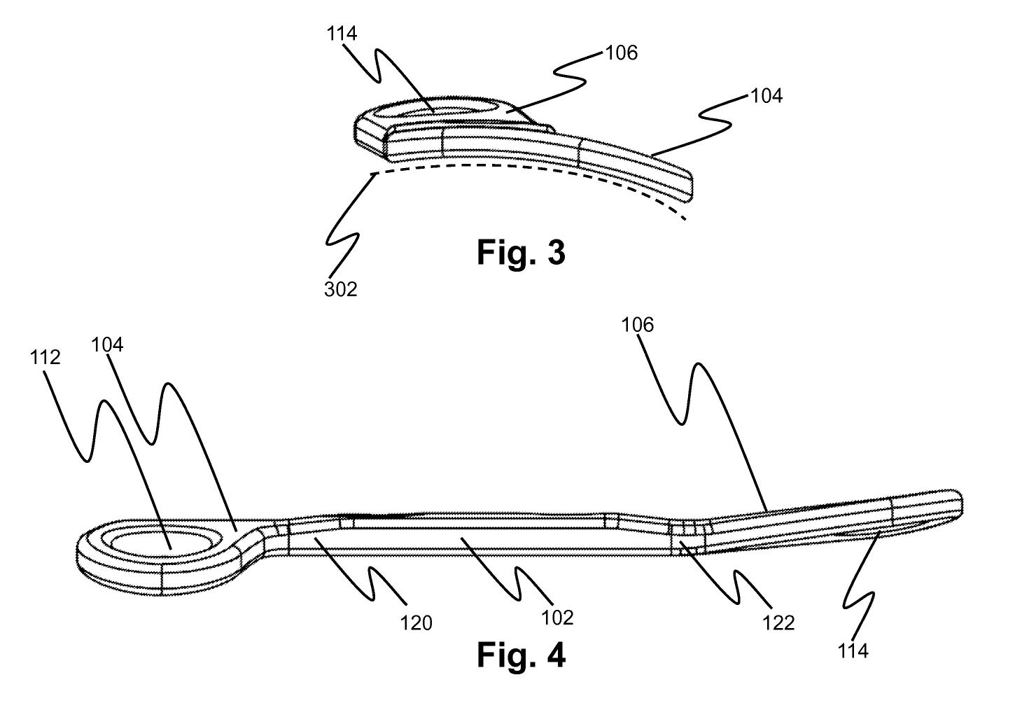

FIG. 1 is a top plan view of an internal plate fixation device 100, according to one or more embodiments of the disclosed subject matter. Plate fixation device 100 includes a straight section (or “middle section”) 102 and anatomically bended legs (or “bent sections”, or “angled legs”) 104 and 106 sized and shaped to encompass a tubular bone as shown, for example, in FIG. 3.

Straight section 102 comprises screw holes (or “screw hole locations”) 108 and 110 for non-locking or locking screws. In some embodiments, screw holes 108 and 110 are not threaded. Alternatively, in other embodiments, screw holes 108 and 110 are threaded.

Legs or bent sections 104 and 106 of the plate 100 provide screw holes 112 and 114, respectively, for locking or non-locking screws. In some embodiments, screw holes 112 and 114 are threaded. Alternatively, in other embodiments, screw holes 112 and 114 are not threaded. In some embodiments, screw holes 112 and 114 have a larger diameter than screw holes 108 and 110.

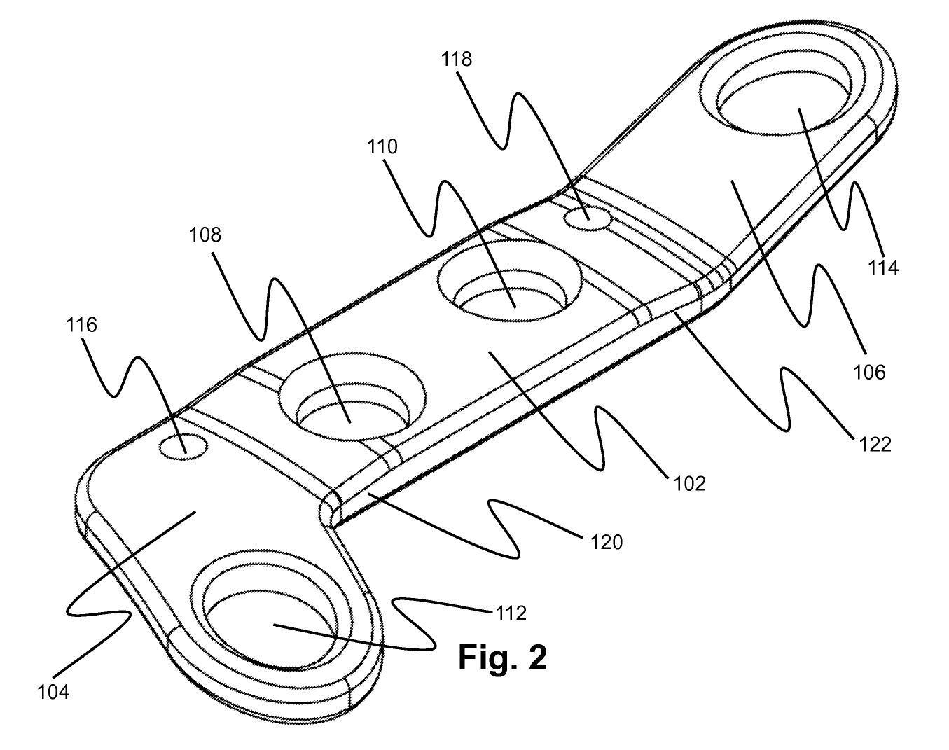

The thickness of the plate 100 can decrease at transfer sections 120 and 122 from a larger thickness of straight section 102 to a smaller thickness of legs or bent sections 104 and 106, respectively, as illustrated, for example, in FIGS. 2 and 4.

Plate 100 can include temporary fixation holes 116 and/or 118 to allow for temporary fixation of the plate 100 to the bone (e.g., a metatarsal bone) before screw fixation. In some embodiments, plate 100 does not include one or both of the temporary fixation holes 116/118.

In some embodiments, screw holes 108 and 110 are top or dorsal screw hole locations and are configured for engagement, angled screw location fixation, to allow for correction fixation once the Mau osteotomy is completed of the first metatarsal bone. In some embodiments, screw hole locations 108 and 110 are configured to provide for concentric angulation and oblique angulation of the screws to ensure screw engagement and fixation of the Mau osteotomy across the Mau osteotomy site, while maintaining the screws are even or flush to the plate 100 surface. In some such embodiments, screw hole locations 108 and 110 are configured to provide for up to 25 degree concentric angulation and oblique angulation of the screws to ensure screw engagement and fixation of the Mau osteotomy across the Mau osteotomy site. Screw hole locations 108 and 110 allow for fixation of the Mau osteotomy when completed in the short length version (i.e., less oblique) of the Mau osteotomy as presented in literature or the long length version (i.e., more oblique) of the Mau osteotomy as presented in literature.

In some embodiments, screw hole locations 112 and 114 are proximal and distal screw hole locations and are for fixation and locking or non-locking into the plate 100 through the metatarsal bone after fixation of the Mau osteotomy using screw holes 108 and 110.

In some embodiments, plate 100 can be altered to adjust to varied anatomy by forcing or bending the proximal and/or distal edges (e.g. 104/106) down to the anatomy after fixation of top or dorsal screws through screw holes 108/110.

Plate 100 can be configured to accommodate various size diameter size locking or non-locking screws that thread into and lock in the plate or that fixate to the plate but do not thread and lock into the plate.

In some embodiments, plate 100 is configured to withstand the weight-bearing capacity of a normal human being and the weight-bearing load displaced to the human metatarsal bone when undergoing the normal walking gait cycle thus making it an option to bear weight immediately after fixation of the plate is applied to the Mau osteotomy, thereby eliminating the concern of stress risers or fractures of the bone dorsally and of dorsal bone displacement.

In some embodiments, plate 100 has a varied thickness as shown, for example, in FIGS. 2 and 4. In some embodiments, plate 100 is low profile so as to not disrupt soft tissue. In some such embodiments, plate 100 is less than 1.8 mm. In some embodiments, the thickness of plate 100 will be slightly thicker dorsally across the osteotomy site to provide strength across the osteotomy site and thinner at both proximal and distal ends to allow for bending of the plate and allow for low profile fixation of the plate to the metatarsal bone. For example, in some embodiments, the straight section 102 of the metallic plate has a thickness of no more than 1.8 millimeters and the bent sections 104 and/or 106 have a thickness of no less than 1.0 millimeters. For example, in some embodiments, plate 100 can have a variable thickness such that plate 100 is adapted to bend at portions oblique to a longitudinal axis of plate 100 (e.g., a longitudinal axis of straight section 102) near where the bent sections 104 and 106 meet the straight section 102.

Plate 100 can be provided in a variety of lengths to accommodate a variety of first metatarsal anatomies. In some embodiments, plate 100 is anatomically designed to the first metatarsal bone and designed to allow completion of the Mau osteotomy and fixation of the Mau osteotomy while in position on the metatarsal bone.

Plate 100 can be made of titanium, titanium or other metal alloy, stainless steel, plastic, resorbable material, medical grade thermoplastic or polymer material, or other suitable material. In some embodiments, plate 100 can be radiolucent and/or osteoconductive. In some embodiments, plate 100 can have one or more portions coated with an osteoconductive material such as, for example, hydroxyapatite (Hap).

FIG. 3 is a back view of the plate fixation device 100 shown in FIG. 1, according to one or more embodiments of the disclosed subject matter. FIG. 3 illustrates anatomically bended legs (or “bent sections”) 104 and 106 sized and shaped to encompass a tubular bone 302 (e.g., a metatarsal bone).

The one or more embodiments shown in FIGS. 1-4 and described above are, for example, adapted for load-bearing or non-load-bearing fixation of the metatarsal correction for a Mau osteotomy correction procedure for hallux valgus correction in the first metatarsal bone in the foot.

In some embodiments, an internal plate fixation device (e.g., 100) includes top or dorsal screw hole locations (e.g., 108/110) configured for engagement, angled screw location fixation, to allow for correction fixation once the Mau osteotomy is completed of the first metatarsal bone. The top or dorsal screw hole locations are configured to provide for up to 25 degree concentric angulation and oblique angulation of the screws to ensure screw engagement and fixation of the Mau osteotomy across the Mau osteotomy site, while maintaining the screws are even or flush to the fixation device surface. The top or dorsal screw hole locations allow for fixation of the Mau osteotomy when completed in the short length version (less oblique) of the Mau osteotomy as presented in literature or the long length version (more oblique) of the Mau osteotomy as presented in literature.

In one or more embodiments, a proximal screw hole location (e.g., 112 or 114) at the leg or bent section of the fixation device will be for fixation and locking or non-locking into the fixation device through the metatarsal bone after fixation of the Mau osteotomy and a distal screw hole location (e.g., 114 or 112) at the leg or bent section end of the fixation device will be for fixation and locking or non-locking into the fixation device through the metatarsal bone after fixation of the Mau osteotomy.

In some embodiments, the fixation device can be altered to adjust to varied anatomy by forcing or bending the proximal and/or distal edges (e.g., 104 and/or 106) down to the anatomy after fixation of the top or dorsal screws that are located in the fixation device.

In some embodiments, the fixation device can accommodate various size diameter size locking or non-locking screws that thread into and lock in the fixation device or that fixate to the fixation device but do not thread and lock into the fixation device.

In some embodiments, the fixation device may withstand the weight-bearing capacity of a normal human being and the weight-bearing load displaced to the human metatarsal bone when undergoing the normal walking gait cycle thus making it an option to bear weight immediately after fixation of the fixation device is applied to the Mau osteotomy, thereby eliminating the concern of stress risers or fractures of the bone dorsally and of dorsal bone displacement.

In some embodiments, the fixation device has a varied thickness. In some embodiments, the fixation device is low profile not to disrupt soft tissue and is less than 1.8 mm. In some embodiments, the thickness will be slightly thicker dorsally across the osteotomy site to provide strength across the osteotomy site and thinner at both proximal and distal ends to allow for bending of the fixation device and allow for low profile fixation of the fixation device to the metatarsal bone. For example, in some embodiments, the fixation device has a thickness of no more than 1.8 millimeters dorsally across the osteotomy site and the proximal/distal ends have a thickness of no less than 1.0 millimeters.

The Mau osteotomy internal fixation device plate can be provided in a variety of lengths to accommodate a variety of first metatarsal anatomies. In some embodiments, the fixation device is anatomically designed to the first metatarsal bone and designed to allow completion of the Mau osteotomy and fixation of the Mau osteotomy while in position on the metatarsal bone.

The fixation device can be made of titanium, titanium alloy, stainless steel, plastic, resorbable material, medical grade thermoplastic or polymer material, any metal alloy or other suitable material.

In some embodiments, the fixation device can be radiolucent and/or osteoconductive. In some embodiments, the fixation device can have one or more portions coated with an osteoconductive material such as, for example, hydroxyapatite (Hap).

In one or more embodiments, a method comprises performing a Mau-type osteotomy cut of a bone to achieve a correction. The method can include using a fixation device such as plate 100 to maintain the correction. The method can include screwing screws through the screw holes 108/110 into the bone to achieve fixation of the correction. After fixation of the correction, the fixation device can be adjusted to the anatomy of the patient, if necessary, by forcing or bending down one or both of the legs 104/106 to the anatomy after fixation of the correction using the screw holes 108/110. The method can include final fixation by screwing screws through the screw holes 112/114 of legs 104/106 into the bone. In some embodiments, the method can also include screwing screws through temporary screw holes 116/118 prior to final fixation.

In one or more first embodiments, a medical fixation device comprises a straight section and first and second anatomically bended legs at opposite ends of the straight section. The straight section can comprise two or more screw holes. The anatomically bended legs can be thinner than the straight section, the thickness of the straight section decreasing at first and second transfer sections adjacent to the anatomically bended legs. The anatomically bended legs can be sized and shaped to encompass a tubular bone for fixation of a hallux valgus correction of a first metatarsal bone of a foot achieved by a Mau-type osteotomy procedure. The anatomically bended legs can each comprise a threaded screw hole.

In one or more of the first embodiments, or any other embodiment, the straight section screw holes can be non-locking screw holes adapted for concentric angulation of non-locking screws to fixate the correction achieved by the Mau-type osteotomy procedure both temporarily and for final fixation through the device to both fixate the correction and firmly hold the device to the metatarsal bone.

In one or more of the first embodiments, or any other embodiment, the screw holes of the anatomically bended legs are distal and proximal locking screw holes having a different diameter than the non-locking screw holes to provide for final fixation and to provide strength/stability of the correction.

In one or more of the first embodiments, or any other embodiment, the straight section screw holes provide angulation of screws to ensure screw engagement and fixation of the Mau-type osteotomy procedure across an osteotomy site, and flush alignment of the screws with respect to a top surface of the device.

In one or more of the first embodiments, or any other embodiment, the device is configured to be adjusted to an anatomy by bending one or both of the anatomically bended legs down to the anatomy after fixation of screws through the straight section screw holes.

In one or more of the first embodiments, or any other embodiment, the device further comprises temporary fixation holes adapted for temporary fixation of the medical fixation device to the first metatarsal bone before screw fixation.

In one or more of the first embodiments, or any other embodiment, the device has a low profile such that the straight section has a thickness of no more than 1.8 millimeters. In one or more of the first embodiments, or any other embodiment, the thickness of the bended legs is no less than 1.0 millimeters.

In one or more of the first embodiments, or any other embodiment, the medical fixation device is metallic. In one or more of the first embodiments, or any other embodiment, the medical fixation device is titanium, titanium alloy, or stainless steel. In one or more of the first embodiments, or any other embodiment, the medical fixation device is a resorbable material or a medical grade thermoplastic or polymer material.

In one or more second embodiments, an internal plate fixation device for a Mau-type osteotomy procedure for hallux valgus correction in a first metatarsal bone of a foot comprises a middle section and anatomically bended legs at opposite ends of the middle section. The anatomically bended legs can be sized and shaped to encompass a tubular bone. The middle section can comprise two non-locking screw holes for fixation of the correction, the non-locking screw holes being configured to provide an angulation of screws to ensure screw engagement and fixation of the correction across an osteotomy site. The anatomically bended legs can each comprise one threaded screw hole for final fixation. The middle section can be thicker than the anatomically bended legs.

In one or more of the second embodiments, or any other embodiment, the middle section screw holes are adapted for up to 25 degree angulation of non-locking screws.

In one or more of the second embodiments, or any other embodiment, the screw holes of the anatomically bended legs have a different diameter than the non-locking screw holes to provide for final fixation and to provide strength/stability of the correction.

In one or more of the second embodiments, or any other embodiment, the middle section screw holes provide angulation of screws to ensure screw engagement and fixation of the Mau-type osteotomy procedure across the osteotomy site, and flush alignment of the screws with respect to a top surface of the device.

In one or more of the second embodiments, or any other embodiment, the device is configured to be adjusted to an anatomy by bending one or both of the anatomically bended legs down to the anatomy after fixation of screws through the middle section screw holes.

In one or more of the second embodiments, or any other embodiment, the device further comprises temporary fixation holes adapted for temporary fixation of the medical fixation device to the first metatarsal bone before screw fixation.

In one or more of the second embodiments, or any other embodiment, the device has a low profile such that the middle section has a thickness of no more than 1.8 millimeters. In one or more of the second embodiments, or any other embodiment, the thickness of the bended legs is no less than 1.0 millimeters. In one or more of the second embodiments, or any other embodiment, the device is titanium, titanium or other metallic alloy, stainless steel, a resorbable material, or a medical grade thermoplastic or polymer material.

In one or more third embodiments, a method comprises performing a Mau-type osteotomy cut of a bone to achieve a correction. A fixation device can be implanted to maintain the correction. The fixation device can comprise a middle section and anatomically bended legs at opposite ends of the middle section. The anatomically bended legs can be sized and shaped to encompass a tubular bone. The middle section can comprise two non-locking screw holes for fixation of the correction. The non-locking screw holes can be configured to provide an angulation of screws to ensure screw engagement and fixation of the correction across an osteotomy site. The anatomically bended legs can each comprise one threaded screw hole for final fixation. The middle section can be thicker than the anatomically bended legs. The method can include inserting screws through the non-locking screw holes into the bone for initial fixation of the correction. After initial fixation of the correction, the fixation device can be adjusted to the anatomy of the bone by bending down one or both of the anatomically bended legs. Screws can be inserted through the threaded screw holes into the bone for final fixation of the correction.

In this application, unless specifically stated otherwise, the use of the singular includes the plural and the use of “or” means “and/or.” Furthermore, use of the terms “including” or “having,” as well as other forms, such as “includes,” “included,” “has,” or “had” is not limiting. Any range described herein will be understood to include the endpoints and all values between the endpoints.

Features of the disclosed embodiments may be combined, rearranged, omitted, etc., within the scope of the invention to produce additional embodiments. Furthermore, certain features may sometimes be used to advantage without a corresponding use of other features.

In the instant specification and claims, the term “class” of patient may refer to size and shape features characteristic of groups of human patients. For example, classes corresponding to foot size, width, and gender of patient may be defined and variously sized and shaped fixation devices manufactured for each. In embodiments, class may refer to a single individual as well.

It is, thus, apparent that there is provided, in accordance with the present disclosure, fixation device for a Mau-type osteotomy procedure. Many alternatives, modifications, and variations are enabled by the present disclosure. While specific embodiments have been shown and described in detail to illustrate the application of the principles of the invention, it will be understood that the invention may be embodied otherwise without departing from such principles. Accordingly, Applicant intends to embrace all such alternatives, modifications, equivalents, and variations that are within the spirit and scope of the present invention.

1. A method for fixation of a hallux valgus correction of a first metatarsal bone of a foot of a patient achieved by a Mau-type osteotomy procedure, the method comprising:
performing the Mau-type osteotomy procedure to achieve a desired hallux valgus correction of the first metatarsal bone of the foot of the patient;
selecting a fixation device from a kit of fixation devices based on a characteristic of the patient and/or the desired correction, the kit comprising two or more different sizes of fixation devices, each size being compatible with a respective predefined class of patient, each fixation device comprising:
a middle section comprising two initial fixation screw holes;
first and second legs at opposite ends of the middle section;
the middle section comprising first and second transfer sections adjacent the first and second legs, respectively, the thickness of the device decreasing at each transfer section from a first thickness to a second thickness of the respective leg;
the device having an initial curvature compatible with its respective predefined class of patient;
the legs each comprising a threaded screw hole;
the initial fixation screw holes being non-locking screw holes adapted for angulation of non-locking screws to fixate the correction achieved by the Mau-type osteotomy procedure,
applying the selected fixation device for initial fixation of the correction;
after initial fixation of the correction, adjusting the selected fixation device to the anatomy of the patient's bone by bending down one or both of the legs, the fixation device having a variable thickness such that the fixation device is adapted to bend at portions oblique to a longitudinal axis of the fixation device to adjust to the anatomy of the patient's bone;
performing final fixation of the correction by applying screws through the threaded screw holes of the legs of the selected fixation device.

2. The method of claim 1, wherein the screw holes of the legs have a larger diameter than that of the non-locking screw holes to provide for final fixation and to provide strength/stability of the correction.

3. The method of claim 1, wherein the initial fixation screw holes provide flush alignment of the non-locking screws with respect to a top surface of the device.

4. The method of claim 1, wherein each fixation device further comprises temporary fixation holes adapted for temporary fixation of the medical fixation device to the first metatarsal bone before screw fixation.

5. The method of claim 1, wherein each fixation device has a low profile such that the middle section has a thickness of no more than 1.8 millimeters.

6. The method of claim 1, wherein the thickness of the legs of each fixation device is no less than 1.0 millimeters.

7. The method of claim 1, wherein at least one of the fixation devices is metallic.

8. The method of claim 1, wherein at least one of the fixation devices is titanium, titanium alloy, or stainless steel.

9. The method of claim 1, wherein at least one of the fixation devices is a resorbable material or a medical grade thermoplastic or polymer material.

10. A medical fixation device, the medical fixation device comprising:
a middle section comprising two initial fixation screw holes;
first and second legs at opposite ends of the middle section;
the middle section comprising first and second transfer sections adjacent the first and second legs, respectively, the thickness of the device decreasing at each transfer section from a first thickness to a second thickness of the respective leg;
the device having an initial curvature compatible with a predefined class of patient for fixation of a hallux valgus correction of a first metatarsal bone of a foot achieved by a Mau-type osteotomy procedure;
the legs each comprising a threaded screw hole;
the initial fixation screw holes being non-locking screw holes adapted for angulation of non-locking screws to fixate the correction achieved by the Mau-type osteotomy procedure.

11. The medical fixation device of claim 10, wherein the initial fixation screw holes have a larger diameter than the non-locking screw holes to provide for final fixation and to provide strength/stability of the correction.

12. The medical fixation device of claim 10, wherein the initial fixation screw holes provide flush alignment of the screws with respect to a top surface of the device.

13. The medical fixation device of claim 10, wherein the device is configured to be adjusted to an anatomy by bending one or both of the legs down to the anatomy after fixation of screws through the straight section screw holes.

14. The medical fixation device of claim 10, further comprising:
temporary fixation holes adapted for temporary fixation of the medical fixation device to the first metatarsal bone before screw fixation.

15. The medical fixation device of claim 10, wherein the device has a low profile such that the middle section has a thickness of no more than 1.8 millimeters.

16. The medical fixation device of claim 15, wherein the thickness of the legs is no less than 1.0 millimeters.

17. The medical fixation device of claim 10, wherein the medical fixation device is metallic.

18. The medical fixation device of claim 17, wherein the medical fixation device is titanium, titanium alloy, or stainless steel.

19. The medical fixation device of claim 10, wherein the medical fixation device is a resorbable material or a medical grade thermoplastic or polymer material.

20. A method, comprising:
performing an osteotomy cut of a bone of a patient to achieve a desired correction;
selecting a fixation device to maintain the correction based on a characteristic of the patient and/or the desired correction, the fixation device comprising:
a middle section comprising two initial fixation screw holes;
legs at opposite ends of the middle section, the legs being sized and shaped to at least partially encompass a tubular bone;
the middle section comprising two non-locking screw holes for fixation of the correction, the non-locking screw holes being configured to provide an angulation of screws to ensure screw engagement and fixation of the correction across an osteotomy site;
the legs each comprising one threaded screw hole for final fixation; and
the middle section being thicker than the legs, the thickness of the device being configured such that the device is adapted to bend at portions oblique to a longitudinal axis of the device,
performing initial fixation of the correction with the selected fixation device;
after initial fixation of the correction, adjusting the selected fixation device to the anatomy of the bone by bending down one or both of the legs;
performing final fixation of the correction with the selected fixation device.
